(19) United States
(12) Patent Application Publication
(10) Pub. No.: US 2022/0177828 A1
(43) Pub. Date: Jun. 9, 2022

(54) APPARATUS AND METHOD FOR CRYSTALLIZATION AND SUPERCRITICAL DRYING OF CULTURE SOLUTION

(71) Applicant: SEOUL NATIONAL UNIVERSITY R&DB FOUNDATION, Seoul (KR)

(72) Inventors: Youn-Woo LEE, Seoul (KR); Wonsu SON, Seoul (KR); Heejeong PARK, Seoul (KR); Sunuk SONG, Seoul (KR); Sina KIM, Seoul (KR); Chanju LEE, Seoul (KR)

(21) Appl. No.: 17/436,511

(22) PCT Filed: Mar. 4, 2020
(86) PCT No.: PCT/KR2020/003058

§ 371 Date: Sep. 3, 2021

(30) Foreign Application Priority Data
Mar. 5, 2019 (KR) 10-2019-0025421

(51) Int. Cl. C12M 1/00; C12N 5/0775

(52) U.S. Cl. CPC C12M 47/02; C12M 47/10; C12N 5/0662

(57) ABSTRACT

An apparatus and a method for crystallization and supercritical drying of culture solution are provided.

According to the embodiments of the present invention, an apparatus for crystallization and supercritical drying of culture solution to obtain target material by drying a culture solution containing a first solvent and the target material dissolved in the first solvent comprises a high pressure container to accommodate the culture solution, a first supply unit connected to the high pressure container to supply a second solvent to the high pressure container, a second supply unit connected to the high pressure container to supply a third solvent to the high pressure container, a precooler disposed adjacent to the first supply unit to precool the second solvent discharged from the first supply unit, and a preheater disposed adjacent to the high pressure container to preheat the second solvent and the third solvent supplied to the high pressure container.

According to the embodiments of the present invention, a method for crystallization and supercritical drying of culture solution to obtain target material by drying a culture solution containing a first solvent and the target material dissolved in the first solvent comprises crystallizing the target material by replacing the first solvent with a second solvent in the culture solution, and changing a phase of the second solvent to a supercritical phase.

### TECHNICAL FIELD

The present invention relates to an apparatus and a method for crystallization and supercritical drying of culture solution.

### BACKGROUND ART

Mesenchymal stem cells (MSCs) are multipotent stem cells that have the ability to differentiate into various mesenchymal cells including bone, cartilage, fat, and muscle cells or into ectodermal cells such as nerve cells. Mesenchymal stem cells are attracting attention as a material for regenerative medicine in that it can achieve successful cell regeneration by suppressing immune rejection.

MSC culture solution for disease treatment is extracted from the cells of the patient in need of treatment and cultured if there is enough time. However, since there is not enough time for culturing in acute patients, the pre-cultured MSC culture solution is frozen and stored, and then thawed and used when necessary. If stored in an aqueous solution that is not frozen, the three-dimensional structure of the material may be collapsed due to amino acid deamination through hydrolysis by moisture, and as a result, the medicinal effect may disappear. In addition, although storage through refrigeration is possible in developed countries with well-established medical infrastructure, there are many people who need treatment using MSC culture solution secretion materials even in developing countries that do not have facilities for culturing or even have no infrastructure for freezing storage but it is difficult to receive the benefits. In order to solve these various problems, drying of the MSC culture is required so that there is no need for freezing.

The conventionally used freeze drying (lyophilization) is a method of sublimating a fluid to be dried into a gas by lowering the temperature to less than a triple point, freezing it, and then reducing the pressure. In the freeze drying, the three-dimensional structure of the protein may be destroyed during the freezing process because the volume of water, which is the fluid to be dried, increases as it freezes. The freeze drying requires a lot of energy and a long time to dry. In addition, the freeze drying process is complicated and difficult, and the moisture in the microstructure of the protein is not completely dried, so the bonds between amino acids may be broken by hydrolysis by the residual moisture.

### DISCLOSURE

### Technical Problem

In order to solve the above mentioned problems, the present invention provides an apparatus for crystallization and supercritical drying of culture solution.

The present invention provides a method for crystallization and supercritical drying of culture solution.

The other objects of the present invention will be clearly understood by reference to the following detailed description and the accompanying drawings.

### Technical Solution

According to the embodiments of the present invention, a method for crystallization and supercritical drying of culture solution to obtain target material by drying a culture solution containing a first solvent and the target material dissolved in the first solvent comprises crystallizing the target material by replacing the first solvent with a second solvent in the culture solution, and changing a phase of the second solvent to a supercritical phase.

According to the embodiments of the present invention, an apparatus for crystallization and supercritical drying of culture solution to obtain target material by drying a culture solution containing a first solvent and the target material dissolved in the first solvent comprises a high pressure container to accommodate the culture solution, a first supply unit connected to the high pressure container to supply a second solvent to the high pressure container, a second supply unit connected to the high pressure container to supply a third solvent to the high pressure container, a precooler disposed adjacent to the first supply unit to precool the second solvent discharged from the first supply unit, and a preheater disposed adjacent to the high pressure container to preheat the second solvent and the third solvent supplied to the high pressure container.

### Advantageous Effects

Crystallization and supercritical drying of the culture solution according to embodiments of the present invention can dry the culture solution while preventing the structural stability and activity of the target material (dried material) dissolved in the culture solution from being denatured. The target material may be crystallized and dried. For example, the crystallization and supercritical drying may be used to crystallize and dry cells or proteins that are sensitive to temperature and need to maintain a microstructure, such as proteins. The crystallization and supercritical drying are superior to freeze drying in terms of process costs such as energy and process time. Mass production is possible by the crystallization and supercritical drying.

### DESCRIPTION OF DRAWINGS

FIG. 1 shows an apparatus for crystallization and supercritical drying of culture solution according to an embodiment of the present invention.

FIGS. 2 and 3 show the ELISA analysis results of the supercritical drying process dried material for each nozzle inner diameter variable. FIG. 2 shows the concentration of VEGF contained in the dried material dissolved at the same concentration for each condition, and FIG. 3 shows the total amount of VEGF for each condition considering the yield.

FIGS. 4 and 5 show a stability test graph of the supercritical drying process dried material through ELISA analysis according to time for each nozzle inner diameter variable. FIG. 4 shows the concentration of VEGF contained in the dried material dissolved at the same concentration for each condition, and FIG. 5 shows the total amount of VEGF for each condition considering the yield.

FIGS. 6 and 7 show the ELISA analysis results of the supercritical drying process dried material for each total flow rate (CO₂+EtOH) variable. FIG. 6 shows the concentration of VEGF contained in the dried material dissolved at the same concentration for each condition, and FIG. 7 shows the total amount of VEGF for each condition considering the yield.

FIGS. 8 and 9 show a stability test graph of the supercritical drying process dried material through ELISA analysis according to time for each total flow rate (CO₂+EtOH) variable. FIG. 8 shows the concentration of VEGF contained in the dried material dissolved at the same concentration for each condition, and FIG. 9 shows the total amount of VEGF for each condition considering the yield.

FIGS. 10 and 11 show the ELISA analysis results of the supercritical drying process dried material for each temperature variable. FIG. 10 shows the concentration of VEGF contained in the dried material dissolved at the same concentration for each condition, and FIG. 11 shows the total amount of VEGF for each condition considering the yield.

FIGS. 12 and 13 show a stability test graph of the supercritical drying process dried material through ELISA analysis according to time for each temperature variable. FIG. 12 shows the concentration of VEGF contained in the dried material dissolved at the same concentration for each condition, and FIG. 13 shows the total amount of VEGF for each condition considering the yield.

FIGS. 14 and 15 show the results of ELISA analysis of the supercritical drying process dried material for each pressure variable. FIG. 14 shows the concentration of VEGF contained in the dried material dissolved at the same concentration for each condition, and FIG. 15 shows the total amount of VEGF for each condition considering the yield.

FIGS. 16 and 17 show a stability test graph of the supercritical drying process dried material through ELISA analysis according to time for each pressure variable. FIG. 16 shows the concentration of VEGF contained in the dried material dissolved at the same concentration for each condition, and FIG. 17 shows the total amount of VEGF for each condition considering the yield.

FIGS. 18 and 19 show the ELISA analysis results of the supercritical drying process dried material for each CO₂/EtOH ratio variable. FIG. 18 shows the concentration of VEGF contained in the dried material dissolved at the same concentration for each condition, and FIG. 19 shows the total amount of VEGF for each condition considering the yield.

FIGS. 20 and 21 show a stability test graph of the supercritical drying process dried material through ELISA analysis according to time for each CO₂/EtOH ratio variable. FIG. 20 shows the concentration of VEGF contained in the dried material dissolved at the same concentration for each condition, and FIG. 21 shows the total amount of VEGF for each condition considering the yield.

FIG. 22 shows the expression patterns of growth factors using a growth factor array of dried materials obtained under optimal conditions for each process.

### BEST MODE

Hereinafter, a detailed description will be given of the present invention with reference to the following embodiments. The purposes, features, and advantages of the present invention will be easily understood through the following embodiments. The present invention is not limited to such embodiments, but may be modified in other forms. The embodiments to be described below are nothing but the ones provided to bring the disclosure of the present invention to perfection and assist those skilled in the art to completely understand the present invention. Therefore, the following embodiments are not to be construed as limiting the present invention.

Terms like ‘first’, ‘second’, etc., may be used to indicate various components, but the components should not be restricted by the terms. These terms are only used to distinguish one element, component, region, layer or section from another region, layer or section. A first element, component, region, layer or section could be termed a second element, component, region, layer or section without departing from the teaching of the embodiments of the present invention.

It will be understood that when an element is referred to as being “connected to” another element, it can be directly connected to the other element or intervening elements may be present therebetween.

The terminology used herein is for the purpose of describing particular embodiments only and is not intended to limit the invention. It is to be understood that the singular forms “a,” “an,” and “the” include plural references unless the context clearly dictates otherwise. It will be further understood that the terms “comprises” or “has,” when used in this specification, specify the presence of stated features, integers, steps, operations, elements, and/or components, but do not preclude the presence or addition of one or more other features, integers, steps, operations, elements, components, and/or groups thereof.

According to the embodiments of the present invention, a method for crystallization and supercritical drying of culture solution to obtain target material by drying a culture solution containing a first solvent and the target material dissolved in the first solvent comprises crystallizing the target material by replacing the first solvent with a second solvent in the culture solution, and changing a phase of the second solvent to a supercritical phase.

The solvent replacing may comprise replacing the first solvent with the second solvent and a third solvent, and the phase changing may comprise removing the third solvent.

The second solvent may have a critical temperature lower than the denaturation temperature of the target material. The third solvent may be mixed with the first solvent and the second solvent. The first solvent may comprise water, the second solvent may comprise at least one of CO₂ and N₂O, and the third solvent may comprise at least one of ethanol, acetone, N-methyl-2-pyrrolidone, and dimethyl sulfoxide.

The target material may maintain structural stability after the supercritical drying. The target material may comprise cells such as stem cells. The target material may comprise a protein.

The method may further comprise vaporizing the second solvent in the supercritical phase.

The method may be carried out at a temperature of 0˜40° C. and a pressure of 35˜500 bar.

According to the embodiments of the present invention, an apparatus for crystallization and supercritical drying of culture solution to obtain target material by drying a culture solution containing a first solvent and the target material dissolved in the first solvent comprises a high pressure container to accommodate the culture solution, a first supply unit connected to the high pressure container to supply a second solvent to the high pressure container, a second supply unit connected to the high pressure container to supply a third solvent to the high pressure container, a precooler disposed adjacent to the first supply unit to precool the second solvent discharged from the first supply unit, and a preheater disposed adjacent to the high pressure container to preheat the second solvent and the third solvent supplied to the high pressure container.

The apparatus may further comprise a capillary tube disposed in the high pressure container to transfer the second solvent and the third solvent into the high pressure container. The apparatus may further comprise a water tank accommodating the high pressure container and the preheater. The apparatus may further comprise a gas-liquid separator connected to the high pressure container to separate a fluid discharged from the high pressure container. The apparatus may further comprise a back pressure regulator disposed between the high pressure container and the gas-liquid separator to control the back pressure of the high pressure container.

The second solvent may have a critical temperature lower than the denaturation temperature of the target material. The third solvent may be mixed with the first solvent and the second solvent. The first solvent may comprise water, the second solvent may comprise at least one of CO₂ and N₂O, and the third solvent may comprise at least one of ethanol, acetone, N-methyl-2-pyrrolidone, and dimethyl sulfoxide.

The target material may maintain structural stability after the supercritical drying. The target material may comprise cells such as stem cells. The target material may comprise a protein.

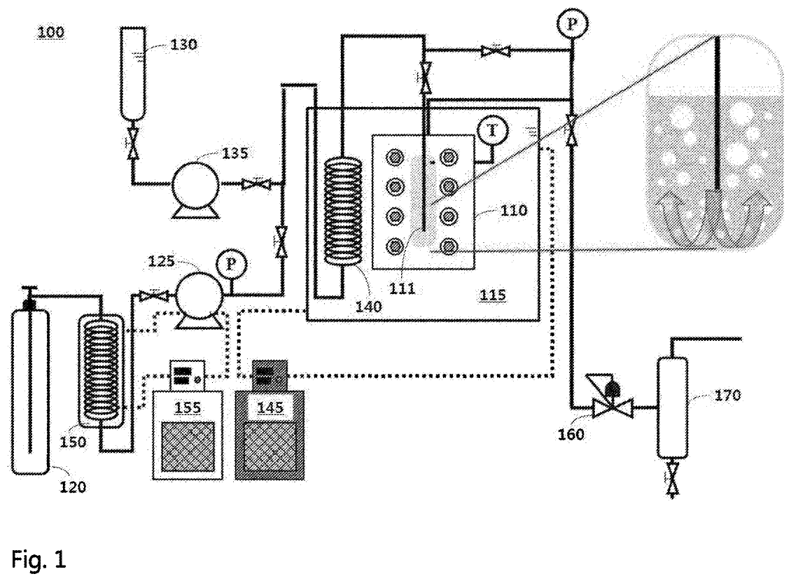

FIG. 1 shows an apparatus for crystallization and supercritical drying of a culture solution according to an embodiment of the present invention.

Referring to FIG. 1, the apparatus 100 for the crystallization and supercritical drying of the culture solution comprises a high pressure container 110, a first supply unit 120, a second supply unit 130, a preheater 140, and a precooler 150 as a supercritical drying apparatus for drying a mixture including a target material and a first solvent (eg, water).

The high pressure container 110 may accommodate, crystallize, and dry the mixture. The first supply unit 120 may be connected to the high pressure container 110 to supply a second solvent (eg, carbon dioxide). The second solvent may be transferred by the first pump 125 disposed between the first supply unit 120 and the high pressure container 110. The second supply unit 130 may be connected to the high pressure container 110 to supply a third solvent (eg, anhydrous ethanol). The third solvent may be transferred by the second pump 135 disposed between the second supply unit 130 and the high pressure container 110.

The preheater 140 may be disposed adjacent to the high pressure container 110 to preheat the second solvent and the third solvent supplied to the high pressure container 110. The heating medium of the preheater 140 may be heated by a heating tank 145 connected to the preheater 140.

The high pressure container 110 and the preheater 140 may be disposed in the water tank 115, and the high pressure container 110 may effectively control the process temperatures of the target material, the first solvent, the second solvent, and the third solvent.

The precooler 150 may be disposed adjacent to the first supply unit 120 to precool the second solvent discharged from the first supply unit 120. The cooling medium of the precooler 150 may be cooled by the cooling tank 155 connected to the precooler 150.

The apparatus 100 for crystallization and supercritical drying of culture solution may comprise a capillary tube 111. The capillary tube 111 is disposed in the high pressure container 110, and transfers the second solvent and the third solvent into the high pressure container 110. The second solvent and the third solvent may be well mixed by the capillary tube 111 and then effectively supplied to the mixture in the high pressure container 110.

The apparatus 100 for crystallization and supercritical drying of culture solution may comprise a gas-liquid separator 170 that is connected to the high pressure container 110 and separates the fluid discharged from the high pressure container 110. The apparatus 100 for crystallization and supercritical drying of culture solution may comprise a back pressure regulator 160 disposed between the high pressure container 110 and the gas-liquid separator 170 to regulate the back pressure of the high pressure container 110.

An example of a method for crystallization and supercritical drying of culture solution using the apparatus 100 for crystallization and supercritical drying of culture solution is as follows.

5 mL of the stem cell culture solution to be dried is placed in the high pressure container 110 of the apparatus 100 for crystallization and supercritical drying of culture solution. Liquid carbon dioxide supercooled to −5° C. is supplied at a flow rate of 10 mL/min through the high pressure pump 125 while maintaining the high pressure container 110 at a temperature of 25° C. and a pressure of 250 bar. At the same time, anhydrous ethanol is supplied at a flow rate of 0.475 mL/min at room temperature using the high pressure pump 135. The supplied mixed solution of carbon dioxide and anhydrous ethanol is preheated to 25° C., and then is sprayed to the stem cell culture solution in the high pressure container 110 through a nozzle (capillary tube) with an inner diameter of 0.02″ and split into small droplets. The mixed solution of carbon dioxide and anhydrous ethanol extracts and removes littly by little the water that dissolves the protein. After crystallizing the material dissolved in the culture solution by completely removing water through the liquid phase replacement step for 160 minutes, the temperature of the high pressure container 110 is raised to 36° C., and the fluid in the high pressure container 110 is replaced with liquid carbon dioxide by stopping the supply of the anhydrous ethanol. After 45 minutes of temperature increase and removal of anhydrous ethanol, the pressure is reduced while maintaining the temperature not to fall below 31° C., and the supercritical carbon dioxide is phase-transformed to gaseous carbon dioxide to dry the crystallized material.

In supercritical drying (SCD), surface tension does not exist at the interface between supercritical fluid-liquid and supercritical fluid-gas, so proteins can be dried while preventing the destruction of the microstructure of the proteins. Since the critical temperature of water, which is a solvent of the stem cell culture solution used in an embodiment of the present invention, is very high as 374° C., when supercritical drying of water is performed, inactivation of proteins by temperature cannot be avoided. Therefore, it is preferable to perform supercritical drying after substitution with a solvent having a low critical temperature. Fluids with a critical temperature lower than the protein denaturation temperature are suitable, and CO₂ is most suitable in terms of physicochemical properties, price, and toxicity. However, in the supercritical drying process, CO₂ alone does not mix with water to replace water, so a cosolvent can be used to quickly replace water. Anhydrous ethanol (Ethyl alcohol anhydrous, EtOH) may be used as the cosolvent. The supercritical drying process comprises a liquid phase step (solvent replacement step), a supercritical phase step, and a decompression step. In the liquid phase step, a mixture of CO₂ and EtOH substitutes water to crystallize the material dissolved in the culture solution. In the supercritical phase step, a phase transition to the supercritical phase occurs through removal of EtOH and temperature rising and pressure rising above the critical point. In the decompression step, the supercritical CO₂ is vaporized. In the decompression step, the pressure is reduced, taking care not to reduce the temperature below the critical temperature. The supercritical drying process has a shorter process time than the freeze drying process and is performed under mild temperature conditions. In addition, since the supercritical drying process does not require freezing of the culture solution unlike freeze drying, it is possible to prevent destruction of the microstructure of proteins due to volume expansion accompanying the crystallization process of water molecules.

### Experimental Examples and Analysis Examples

The stem cell culture solution was crystallized and dried under various conditions using supercritical drying (SCD), and the dried solid was analyzed. As comparative examples, the stem cell culture solution was dried using freeze drying (FD), and the dried solid was analyzed. The freeze drying was performed at a temperature of −80° C. and a pressure of 5 mTorr.

The dried solid was dissolved in water at a constant concentration and analyzed using ELISA (Enzyme Linked Immunosorbent Assay). The degree of structural maintenance of specific proteins was measured and the results for each condition were compared. When the yield was 100%, the mass value was 57.5 mg, and water was added according to the mass of the dried material obtained in each process so that 57.5 mg per 5 mL was dissolved, and ELISA analysis was performed. The corresponding mass value was obtained through the average value of the mass of the dried material obtained through several times of oven drying. Oven drying is performed at a high temperature above 60° C., and destruction of the microstructure is performed as evaporation drying proceeds. Therefore, the dried material thus obtained loses its activity. However, at the temperature, the organic matter contained in the culture solution is not decomposed into carbon dioxide and water, so there is no need to consider the mass loss of the dried material. Therefore, only the mass of the dried material obtained from oven drying was measured without ELISA analysis to calculate the yield of other drying processes.

By dissolving in water in proportion to the mass of the dried material, the dried material obtained under each condition can be made to the same concentration. The VEGF (Vascular Endothelial Growth Factor) concentration value obtained through analysis of the dried material dissolved at the same concentration is a VEGF that maintains its structure except for VEGF that is lost or inactivated through structural modification, etc. during the process of the corresponding conditions on the basis of 100% yield. It can be determined which process conditions are optimal conditions for maintaining the structure of VEGF through the relative values of the obtained VEGF concentration values. Although the VEGF concentration value obtained in each process is important, it is also important to calculate the total amount of target protein obtained in consideration of the yield of each process. Since the concentration of the dried material was adjusted so that 57.5 mg per 5 mL was dissolved, when calculating the total amount of VEGF considering the yield, it can be calculated by multiplying the VEGF concentration value obtained through ELISA analysis by the mass (mg)/(57.5 mg/5 mL) of the dried material.

When the protein thus obtained is used commercially, it is exposed to an environment in which moisture is present. Therefore, it is important not only how long the obtained dried material maintains the structure of the protein, but also how long it maintains the structure in the aqueous solution. The aqueous solution of the dried material used for ELISA analysis was stored at 4° C., and ELISA analysis was performed again 2 weeks later to obtain the concentration of VEGF contained in the dried material and calculate the structural retention rate.

The structural retention rate (%) of VEGF after 2 weeks=(the concentration (pg/mL) of VEGF after 2 weeks/the concentration (pg/mL) of VEGF immediately after exposure to an aqueous solution)×100(%)

Comparison of Protein Structure Maintenance and Stability According to Nozzle Inner Diameter Variables

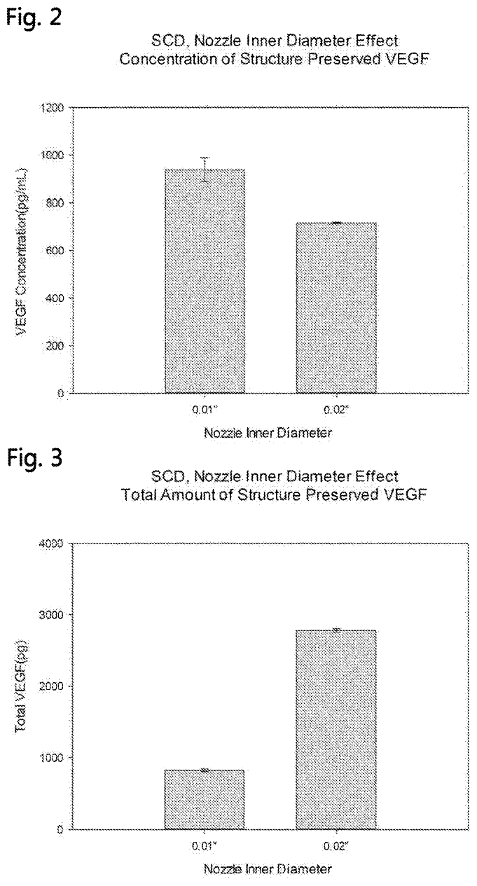

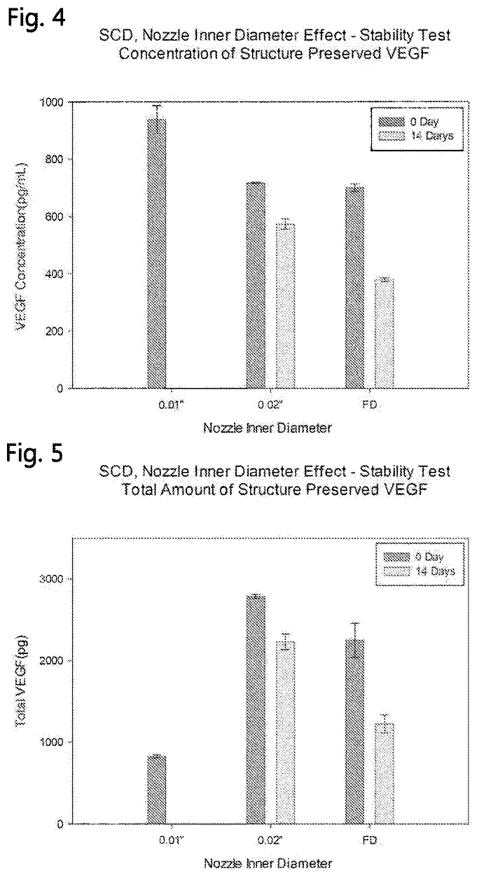

Experiments were performed by changing only the nozzle inner diameter condition (0.01″, 0.02″) in the supercritical drying process, and the experimental conditions and results are shown in Tables 1 to 3 and FIGS. 2 to 5. Table 1 shows the experimental results for each nozzle inner diameter variable in the supercritical drying process, Table 2 shows the ELISA analysis results of the supercritical drying process dried material according to the time for each nozzle inner diameter variable, and Table 3 shows the VEGF structure retention rate (%) for each nozzle inner diameter when the critical drying process dried material was exposed to an aqueous solution for 14 days.

TABLE 1

| | Nozzle | | | CO₂ | EtOH | CO₂/EtOH | Liquid phase | Supercritical | | |
|---|---|---|---|---|---|---|---|---|---|---|
| | inner | Temperature | Pressure | flow rate | flow rate | ratio(v/v, | step time | phase step | Mass (mg) | |
| Sample | diameter | (° C.) | (bar) | (mL/min) | (mL/min) | mL) | (min) | time (min) | Yield (%) | |
| SCD1 | 0.01″ | 15 | 150 | 10 | 0.850 | 10/0.350 | 200 | 55 | a | b |
| | | | | | | | | | 9.8 | 10.4 |
| | | | | | | | | | 17.0 | 18.1 |
| SCD2 | 0.02″ | | | | | | 290 | | a | b |
| | | | | | | | | | 44.1 | 45.2 |
| | | | | | | | | | 78.7 | 78.6 |

| | | | |
|---|---|---|---|
| FD | — | a | b |
| | | 39.6 | 34.2 |
| | | 68.9 | 59.5 |
| Control | — | a | — |
| | | 57.5 | |
| | | 100 | |

TABLE 2

| | 0 Day | | | | 14 Days | | | |
|---|---|---|---|---|---|---|---|---|
| | VEGF concentration | | VEGF total amount | | VEGF concentration | | VEGF total amount | |
| Sample | (pg/mL) | | (pg) | | (pg/mL) | | (pg) | |
| SCD1 | a | b | a | b | a | b | a | b |
| | 988 | 889 | 842 | 804 | 0 | 0 | 0 | 0 |
| SCD2 | a | b | a | b | a | b | a | b |
| | 719 | 714 | 2758 | 2806 | 556 | 592 | 2131 | 2327 |
| FD | a | b | a | b | a | b | a | b |
| | 714 | 687 | 2457 | 2042 | 387 | 373 | 1331 | 1110 |
| Control | a | — | a | — | a | — | a | — |
| | 887 | | 4484 | | 799 | | 3994 | |

TABLE 3

| Sample | SCD1 | SCD2 | FD |
|---|---|---|---|
| VEGF structure | 0 | 80.1 | 54.3 |
| retention rate (%) | | | |

Referring to Tables 1 to 3 and FIGS. 2 to 5, the concentration of VEGF decreased as the nozzle inner diameter became smaller. The smaller the nozzle inner diameter is, the smaller droplet is formed when the mixed fluid of CO₂ and EtOH is sprayed, and the surface area in contact with H₂O increases, thereby improving the mass transfer rate and reducing the time of the liquid phase step of replacing the solvent. When using a 0.01″ inner diameter nozzle, the droplets are too small to break and an emulsion is formed. The emulsion formed in this way does not disappear until it exits the supercritical dryer, so a significant amount of H₂O in which the protein is dissolved escapes without being mixed with CO₂ and EtOH, so the yield may decrease. Therefore, for the total amount of VEGF, a nozzle with an inner diameter of 0.02″ is advantageous.

The sample dried under the condition of a nozzle with an inner diameter of 0.01″ inner diameter nozzle did not maintain the VEGF structure in aqueous solution. This is considered to be due to the difference in shear stress applied to the protein. The shear stress is proportional to the flow rate of the continuous phase, and in the case of a 0.01″ inner diameter nozzle, the flow rate at the moment of injection is 4 times higher than that of a 0.02″ inner diameter nozzle, so the shear stress is also 4 times higher. When exposed to such a 0.01″ inner diameter nozzle environment, the structure of VEGF can be dried in a state in which it is significantly damaged. In the case of supercritical drying using a 0.02″ inner diameter nozzle, the structural retention rate is quite high. The dried material of the freeze drying process has a significantly lower structural retention rate in the aqueous solution, so the total amount of VEGF decreases significantly after 14 days than the dried material of the supercritical drying process.

Experiments in the subsequent supercritical drying process were performed using a 0.02″ inner diameter nozzle, which is more advantageous in terms of total amount and structural retention rate of VEGF.

Comparison of Protein Structure Maintenance and Stability According to CO₂ Flow Rate+EtOH Flow Rate

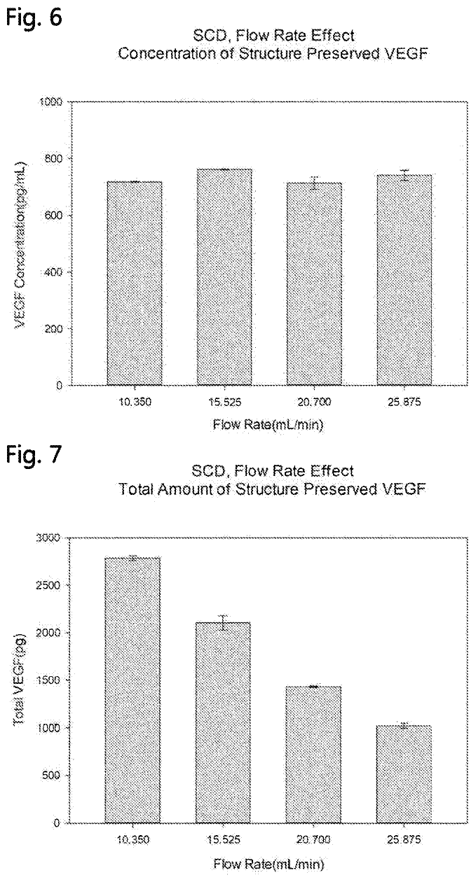

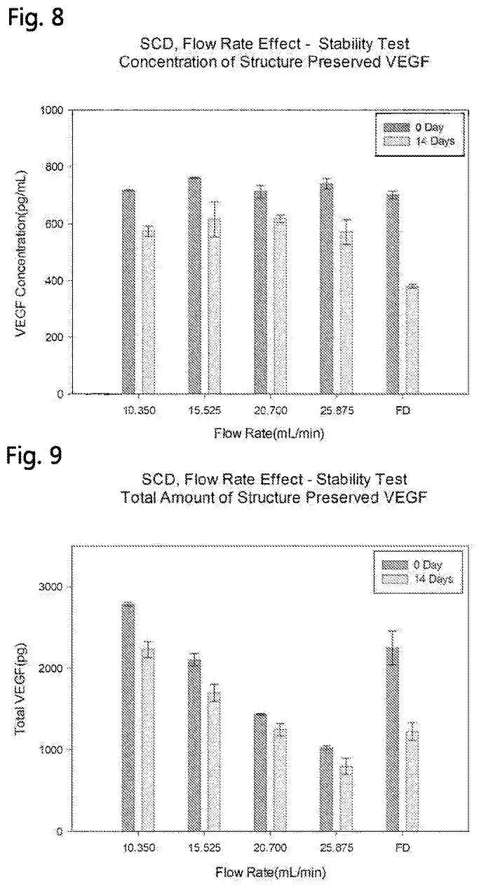

Experiments were performed by changing only the sum of the CO₂ flow rate and the EtOH flow rate (10.350, 15.525, 20.700, 25.875 mL/min) in the supercritical drying process, and the experimental conditions and results are shown in Tables 4 to 6 and FIGS. 6 to 9. Table 4 shows the experimental results for each total flow rate (CO₂+EtOH) in the supercritical drying process, Table 5 shows the ELISA analysis results of the supercritical drying process dried material according to time for each total flow rate (CO₂+EtOH), and Table 6 shows the VEGF structure retention rate (%) for each total flow rate (CO₂+EtOH) when the critical drying process dried material was exposed to an aqueous solution for 14 days.

TABLE 4

| | Nozzle | | | CO₂ | EtOH | CO₂/EtOH | Liquid phase | Supercritical | | |
|---|---|---|---|---|---|---|---|---|---|---|
| | inner | Temperature | Pressure | flow rate | flow rate | ratio(v/v, | step time | phase step | Mass (mg) | |
| Sample | diameter | (° C.) | (bar) | (mL/min) | (mL/min) | mL) | (min) | time (min) | Yield (%) | |
| SCD2 | 0.02″ | 15 | 150 | 10 | 0.350 | 10/0.350 | 290 | 55 | a | b |
| | | | | | | | | | 44.1 | 45.2 |
| | | | | | | | | | 76.7 | 78.6 |
| SCD3 | | | | 15 | 0.525 | | 150 | | a | b |
| | | | | | | | | | 33 | 30.6 |
| | | | | | | | | | 57.4 | 53.2 |
| SCP4 | | | | 20 | 0.700 | | 100 | | a | b |
| | | | | | | | | | 24.0 | 22.3 |
| | | | | | | | | | 41.7 | 38.8 |
| SCD5 | | | | 25 | 0.875 | | 70 | | a | b |
| | | | | | | | | | 15.1 | 16.7 |
| | | | | | | | | | 26.3 | 29.0 |

| | | | |
|---|---|---|---|
| FD | — | a | b |
| | | 35.5 | 39.7 |
| | | 61.7 | 69.0 |
| Control | — | a | — |
| | | 57.5 | |
| | | 100 | |

TABLE 5

| | 0 Day | | | | 14 Days | | | |
|---|---|---|---|---|---|---|---|---|
| | VEGF concentration | | VEGF total amount | | VEGF concentration | | VEGF total amount | |
| Sample | (pg/mL) | | (pg) | | (pg/mL) | | (pg) | |
| SCD2 | a | b | a | b | a | b | a | b |
| | 719 | 714 | 2758 | 2806 | 556 | 592 | 2131 | 2327 |
| SCD3 | a | b | a | b | a | b | a | b |
| | 759 | 762 | 2177 | 2029 | 554 | 677 | 1591 | 1801 |
| SCD4 | a | b | a | b | a | b | a | b |
| | 690 | 734 | 1440 | 1424 | 632 | 604 | 1319 | 1171 |
| SCD5 | a | b | a | b | a | b | a | b |
| | 758 | 722 | 996 | 1048 | 528 | 614 | 693 | 892 |
| FD | a | b | a | b | a | b | a | b |
| | 714 | 687 | 2457 | 2042 | 387 | 373 | 1331 | 1110 |
| Control | a | — | a | — | a | — | a | — |
| | 897 | | 4484 | | 799 | | 3994 | |

TABLE 6

| Sample | SCD2 | SCD3 | SCD4 | SCD5 | FD |
|---|---|---|---|---|---|
| VEGF structure | 80.1 | 80.9 | 86.8 | 77.2 | 54.3 |
| retention rate (%) | | | | | |

Referring to Tables 4 to 6 and FIGS. 6 to 9, the concentration of VEGF was similar regardless of the total flow rate. This is because there is no difference in the temperature and pressure conditions of CO₂, so there is no difference in the acidic environment to which the protein is exposed. However, the yield decreases as the total flow rate increases. When the total flow rate increases, fine H₂O droplets that are not mixed with the CO₂+EtOH mixed fluid are generated by the jet stream generated when the fluid is sprayed from the nozzle, and the protein is dissolved in the droplets. The droplet rises according to the flow of the fluid, and as the total flow rate increases, the flow velocity increases, and when the flow velocity increases, the yield decreases sharply because the fine H₂O droplets in which the protein is significantly dissolved exit the supercritical dryer. Therefore, a low flow rate is advantageous for the total amount of VEGF.

The difference in the total flow rate does not significantly affect the degree of maintaining the VEGF structure in the aqueous phase of the dried sample. The structure retention rate is the highest when the total flow rate is 20.700 mL/min, but it is not significantly different from other conditions, and a low flow rate condition is preferable because it is disadvantageous compared to 10.350 mL/min in yield. The dried material of the freeze drying process has a significantly lower structural retention rate in the aqueous solution, so the total amount of VEGF decreases significantly after 14 days than the dried material of the supercritical drying process.

Subsequent supercritical drying process experiments were performed with a total flow rate of 10.350 mL/min, which is more favorable for the total amount of VEGF.

Comparison of Protein Structure Maintenance and Stability According to Temperature

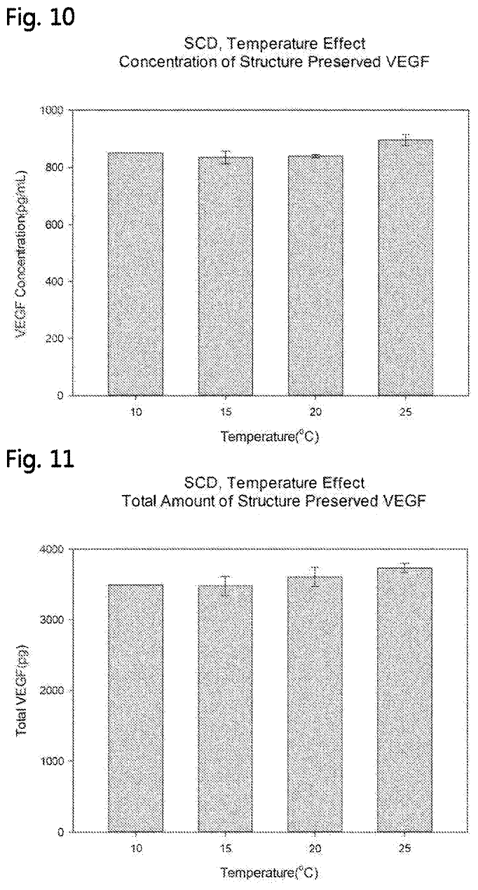

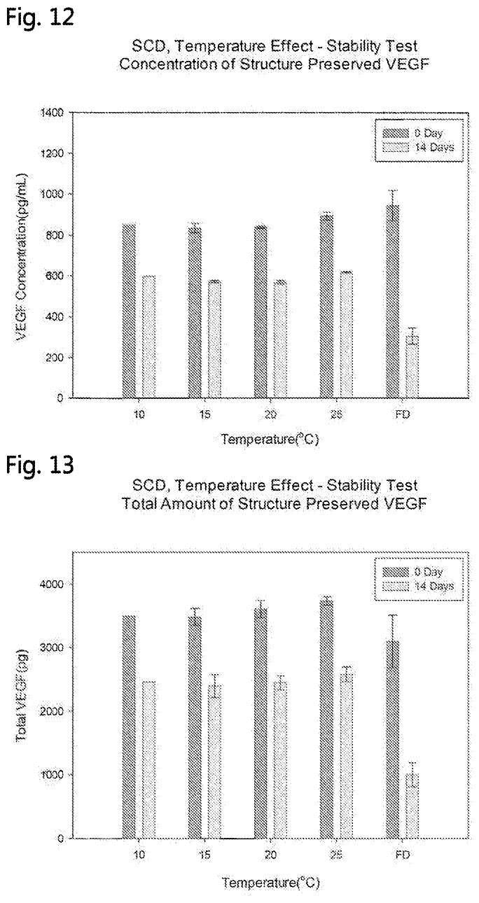

Experiments were performed by changing only the temperature (10, 15, 20, 25° C.) in the supercritical drying process, and the experimental conditions and results are shown in Tables 7 to 9 and FIGS. 10 to 13. Table 7 shows the experimental results for each temperature variable in the supercritical drying process, Table 8 shows the ELISA analysis results of the supercritical drying process dried material according to time for each temperature condition, and Table 9 shows the VEGF structure retention rate (%) for each temperature variable when the critical drying process dried material was exposed to an aqueous solution for 14 days.

TABLE 7

| | Nozzle | | | CO₂ | EtOH | CO₂/EtOH | Liquid phase | Supercritical | | |
|---|---|---|---|---|---|---|---|---|---|---|
| | inner | Temperature | Pressure | flow rate | flow rate | ratio(v/v, | step time | phase step | Mass (mg) | |
| Sample | diameter | (° C.) | (bar) | (mL/min) | (mL/min) | mL) | (min) | time (min) | Yield (%) | |
| SCD6 | 0.02″ | 10 | 150 | 10 | 0.350 | 10/0.350 | 360 | 60 | a | — |
| | | | | | | | | | 47.3 | |
| | | | | | | | | | 82.3 | |
| SCD7 | | 15 | | | | | 290 | 55 | a | b |
| | | | | | | | | | 44.9 | 51.2 |
| | | | | | | | | | 78.1 | 89.0 |
| SCD8 | | 20 | | | | | 270 | 50 | a | b |
| | | | | | | | | | 51.0 | 47.9 |
| | | | | | | | | | 88.7 | 83.3 |
| SCD9 | | 25 | | | | | 255 | 45 | a | b |
| | | | | | | | | | 46.1 | 49.9 |
| | | | | | | | | | 80.2 | 86.8 |

| | | | |
|---|---|---|---|
| FD | — | a | b |
| | | 33.7 | 44.2 |
| | | 58.6 | 76.9 |
| Control | — | a | — |
| | | 57.5 | |
| | | 100 | |

TABLE 8

| | 0 Day | | | | 14 Days | | | |
|---|---|---|---|---|---|---|---|---|
| | VEGF concentration | | VEGF total amount | | VEGF concentration | | VEGF total amount | |
| Sample | (pg/mL) | | (pg) | | (pg/mL) | | (pg) | |
| SCD6 | a | — | a | — | a | — | a | — |
| | 849 | | 3493 | | 597 | | 2456 | |
| SCD7 | a | b | a | b | a | b | a | b |
| | 857 | 812 | 3345 | 3614 | 567 | 578 | 2213 | 2572 |
| SCD8 | a | b | a | b | b | b | a | b |
| | 843 | 833 | 3740 | 3470 | 577 | 560 | 2558 | 2333 |
| SCD9 | a | b | a | b | a | b | a | b |
| | 914 | 876 | 3664 | 3799 | 614 | 622 | 2460 | 2697 |
| FD | 3 | b | a | b | a | b | a | b |
| | 870 | 1017 | 2886 | 3511 | 263 | 343 | 813 | 1185 |
| Control | a | — | a | — | a | — | a | — |
| | 1022 | | 6223 | | 511 | | 2554 | |

TABLE 9

| Sample | SCD6 | SCD7 | SCD8 | SCD9 | FD |
|---|---|---|---|---|---|
| VEGF structure | 70.3 | 68.6 | 67.8 | 69.0 | 32.1 |
| retention rate (%) | | | | | |

Referring to Tables 7 to 9 and FIGS. 10 to 13, the concentration of VEGF was not significantly affected by temperature. This is because the higher the temperature is, the higher the solubility of CO₂ in H₂O in the liquid phase step is, and the protein is exposed to the acidic environment of low pH, but the high temperature causes rapid substitution and decreases the exposure time. The highest concentration was shown at 25° C. In terms of yield, there were no significant differences according to temperature conditions. It is considered that the yield was similar because the flow conditions affecting the formation of fine H₂O droplets by the jet stream did not change. The total amount of VEGF was high at 25° C.

The temperature difference does not significantly affect the retention rate of the VEGF structure in the aqueous solution of the dried sample. As described above, the higher the temperature is, the higher the solubility of CO₂ in H₂O in the liquid phase step is, and the protein is exposed to an acidic environment of low pH, but the exposure time itself decreases due to rapid substitution by high temperature. The dried material of the freeze drying process has a significantly lower structural retention rate in the aqueous solution, so the total amount of VEGF decreases significantly after 14 days than the dried material of the supercritical drying process.

Subsequent supercritical drying process experiments were observed to be slightly higher in the total amount of VEGF, and were carried out under a temperature condition of 25° C. which is close to room temperature and is advantageous in terms of energy consumption.

Comparison of Protein Structure Maintenance and Stability According to Pressure

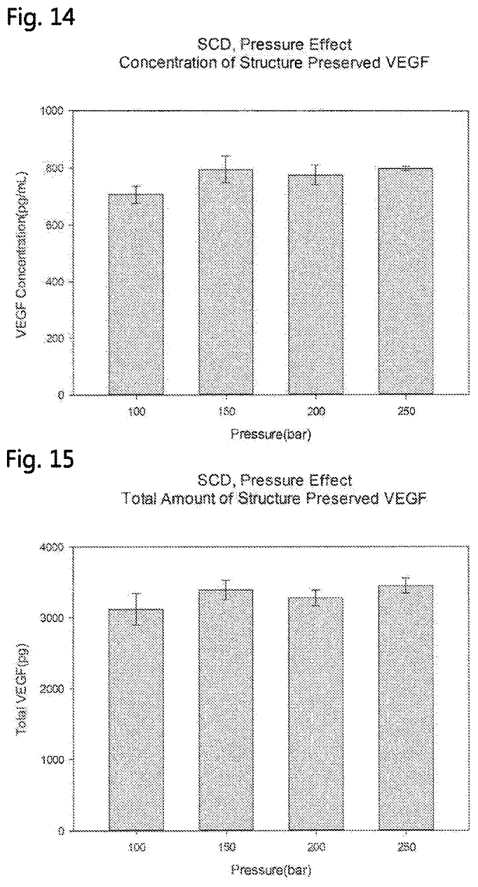

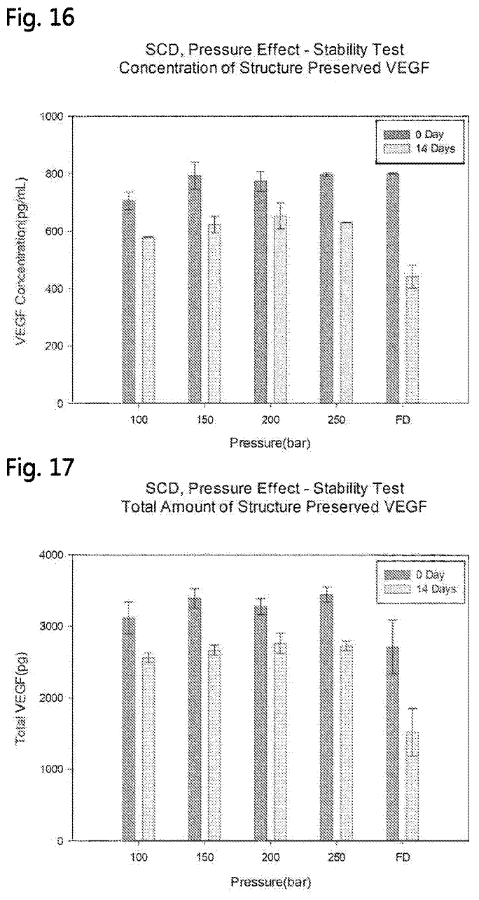

Experiments were performed by changing only the pressure (100, 150, 200, 250 bar) in the supercritical drying process, and the experimental conditions and results are shown in Tables 10 to 12 and FIGS. 14 to 17. Table 10 shows the experimental results for each pressure variable in the supercritical drying process, Table 8 shows the ELISA analysis results of the supercritical drying process dried material according to time for each pressure condition, and Table 9 shows the VEGF structure retention rate (%) for each pressure variable when the critical drying process dried material was exposed to an aqueous solution for 14 days.

TABLE 10

| | Nozzle | | | CO₂ | EtOH | CO₂/EtOH | Liquid phase | Supercritical | | | |
|---|---|---|---|---|---|---|---|---|---|---|---|
| | inner | Temperature | Pressure | flow rate | flow rate | ratio (v/v, | step time | phase step | Mass (mg) | | |
| Sample | diameter | (° C.) | (bar) | (mL/min) | (mL/min) | mL) | (mL) | time (min) | Yield (%) | | |
| SCD10 | 0.02″ | 25 | 100 | 10 | 0.350 | 10/0.350 | 255 | 45 | a | b | — |
| | | | | | | | | | 52.3 | 49.3 | |
| | | | | | | | | | 91.0 | 85.7 | |
| SCD11 | | | 150 | | | | | | a | b | |
| | | | | | | | | | 50.2 | 48.8 | |
| | | | | | | | | | 87.3 | 84.0 | |
| SCD12 | | | 200 | | | | | | a | b | c |
| | | | | | | | | | 48.9 | 47.6 | 49.6 |
| | | | | | | | | | 85.0 | 83.8 | 86.3 |
| SCD13 | | | 250 | | | | | | a | b | — |
| | | | | | | | | | 48.7 | 50.9 | |
| | | | | | | | | | 84.7 | 88.5 | |

| | | | |
|---|---|---|---|
| FD | — | a | b |
| | | 33.7 | 44.2 |
| | | 58.6 | 76.9 |

| | | | |
|---|---|---|---|
| Control | — | a | — |
| | | 57.5 | |
| | | 100 | |

TABLE 11

| | 0 Day | | | | | | 14 Days | | | | | |
|---|---|---|---|---|---|---|---|---|---|---|---|---|
| | VEGF concentration | | | VEGF total amount | | | VEGF concentration | | | VEGF total amount | | |
| Sample | (pg/mL) | | | (pg) | | | (pg/mL) | | | (pg) | | |
| SCD10 | a | b | — | a | b | — | a | b | — | a | b | — |
| | 735 | 676 | | 3342 | 2898 | | 578 | 582 | | 2628 | 2493 | |
| SCD11 | a | b | | a | b | | a | b | | a | b | |
| | 746 | 840 | | 3256 | 3529 | | 594 | 652 | | 2592 | 2740 | |
| SCD12 | a | b | c | a | b | c | a | b | c | a | b | c |
| | 798 | 800 | 735 | 3393 | 3311 | 3126 | 661 | 704 | 595 | 2811 | 2915 | 2567 |
| SCD13 | a | b | — | a | b | — | a | b | — | a | b | — |
| | 790 | 803 | | 3344 | 3554 | | 629 | 631 | | 2665 | 2795 | |
| FD | a | b | | a | b | | a | b | | a | b | |
| | 796 | 803 | | 2339 | 3087 | | 402 | 482 | | 1179 | 1851 | |

| | | | | | | | | |
|---|---|---|---|---|---|---|---|---|
| Control | a | — | a | — | a | — | a | — |
| | 1075 | | 5373 | | 805 | | 4034 | |

TABLE 12

| Sample | SCD10 | SCD11 | SCD12 | SCD13 | FD |
|---|---|---|---|---|---|
| VEGF structure | 82.2 | 78.6 | 84.4 | 79.2 | 55.2 |
| retention rate (%) | | | | | |

Referring to Tables 10 to 12 and FIGS. 14 to 17, the concentration of VEGF was not significantly affected by the pressure except when it was 100 bar. The reason for showing a slightly lower VEGF concentration at 100 bar than other pressure conditions is because in order to reach the supercritical phase, temperature and pressure conditions must be higher than the critical temperature and the critical pressure, and the critical pressure of the mixture of CO₂ and EtOH is higher than 100 bar. That is, since the pressure in the supercritical phase step was not sufficiently applied, the supercritical drying was not performed properly at the time of decompression. This may adversely affect the structure maintenance of VEGF. The yield was not significantly affected by the pressure conditions. It is considered that the yield was similar because the flow conditions affecting the formation of fine H₂O droplets by the jet stream did not change. The total amount of VEGF was similar for the rest of the pressure conditions except when it was 100 bar.

The structure retention rate of VEGF in the aqueous phase of the dried sample is not significantly affected by pressure. In the supercritical drying process, proteins are exposed to long-term shear stress due to the continuous delivery of mixture of water and ethanol. Therefore, even if the shear stress is not sufficient to irreversibly denature the protein structure at a time, structural damage proceeds if the shear stress is raised above the shear stress sufficient to cause damage little by little. In the 0.01″ inner diameter nozzle test, the shear stress value rises higher than that and damage may proceed, but in the 0.02″ inner diameter nozzle test of 250 bar or less, the shear stress like that does not occur. The dried material of the freeze drying process has a significantly lower structural retention rate in the aqueous solution, so the total amount of VEGF decreases significantly after 14 days than the dried material of the supercritical drying process. The dried material through supercritical drying showed a higher VEGF structure retention rate than the sample without any drying process (Control), which is thought to have been influenced by the presence or absence of additives that may accelerate the rate of protein hydrolysis in the culture solution.

Since the experimental results of 150, 200, and 250 bar conditions were quite similar, subsequent supercritical drying process experiments were performed under 250 bar conditions with a narrower experimental error range.

Comparison of Protein Structure Maintenance and Stability According to CO₂/EtoH Ratio

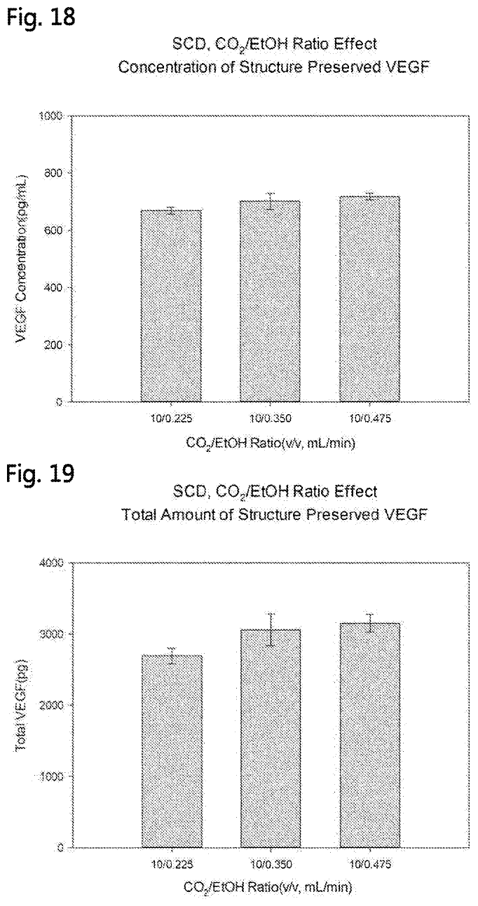

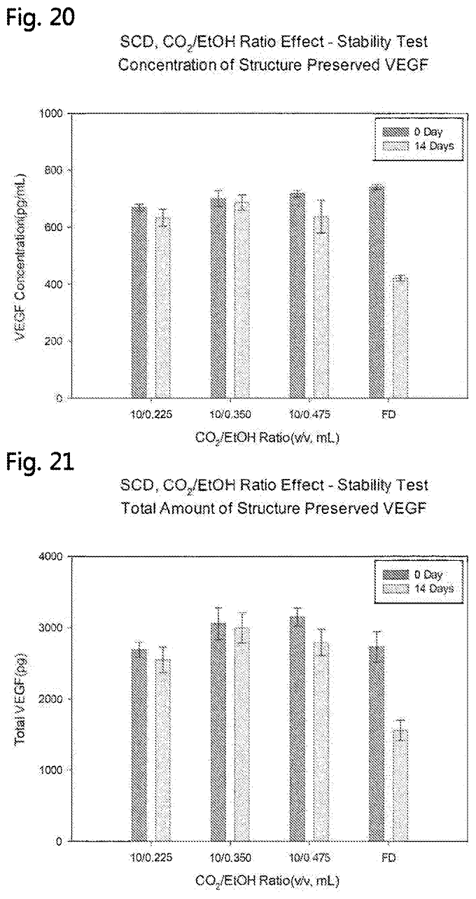

Experiments were performed by changing only the CO₂/EtoH ratio (10/0.225, 10/0.350, 10/0.475 mL/mL) in the supercritical drying process, and the experimental conditions and results are shown in Tables 13 to 15 and FIGS. 18 to 21. Table 13 shows the experimental results for each CO₂/EtoH ratio variable in the supercritical drying process, Table 8 shows the ELISA analysis results of the supercritical drying process dried material according to time for each CO₂/EtoH ratio condition, and Table 9 shows the VEGF structure retention rate (%) for each CO₂/EtoH ratio variable when the critical drying process dried material was exposed to an aqueous solution for 14 days.

TABLE 13

| | Nozzle | | | CO₂ | EtOH | CO₂/EtOH | Liquid phase | Supercritical | | | Residual |
|---|---|---|---|---|---|---|---|---|---|---|---|
| | inner | Temperature | Pressure | flow rale | flow rate | ratio(v/v, | step time | phase step | Mass (mg) | | solvent |
| Sample | diameter | (° C.) | (bar) | (mL/min) | (mL/min) | mL) | (min) | time (min) | Yieid (%) | | (ppm) |
| SCD14 | 0.02″ | 25 | 250 | 10 | 0.225 | 10/0.225 | 435 | 45 | a | b | — |
| | | | | | | | | | 45.2 | 47.2 | |
| | | | | | | | | | 78.6 | 82.1 | |
| SCD15 | | | | | 0.350 | 10/0.350 | 255 | | a | b | — |
| | | | | | | | | | 51.8 | 48.4 | |
| | | | | | | | | | 90.1 | 84.2 | |
| SCD16 | | | | | 0.475 | 10/0.475 | 160 | | a | b | 1.62 |
| | | | | | | | | | 51.7 | 49.3 | |
| | | | | | | | | | 89.9 | 85.7 | |

| | | | | |
|---|---|---|---|---|
| FD | — | a | b | — |
| | | 45.2 | 39.5 | |
| | | 78.6 | 68.7 | |
| Control | — | a | — | — |
| | | 57.5 | | |
| | | 100 | | |

TABLE 14

| | 0 Day | | | | 14 Days | | | |
|---|---|---|---|---|---|---|---|---|
| | VEGF concentration | | VEGF total amount | | VEGF concentration | | VEGF total amount | |
| Sample | (pg/mL) | | (pg) | | (pg/ml) | | (pg) | |
| SCD14 | a | b | a | b | a | b | a | b |
| | 657 | 681 | 2583 | 2797 | 603 | 664 | 2370 | 2725 |
| SCD15 | a | b | a | b | a | b | a | b |
| | 728 | 673 | 3279 | 3833 | 713 | 661 | 3210 | 2782 |
| SCD16 | a | b | a | b | a | b | a | b |
| | 728 | 706 | 3275 | 3026 | 580 | 694 | 2610 | 2976 |
| FD | a | b | a | b | a | b | a | b |
| | 749 | 732 | 2944 | 2515 | 432 | 412 | 1697 | 1415 |
| Control | a | — | a | — | a | — | a | — |
| | 867 | | 4333 | | 677 | | 3383 | |

TABLE 15

| Sample | SCD14 | SCD15 | SCD16 | FD |
|---|---|---|---|---|
| VEGF structure | 94.6 | 98.0 | 88.9 | 57.0 |
| retention rate (%) | | | | |

Referring to Tables 13 to 15 and FIGS. 18 to 21, the concentration of VEGF increases slightly as the EtOH ratio increases, but does not show a significant difference. This is because, since the process temperature and pressure are the same in each condition experiment, the amount of CO₂ saturated in H₂O is the same to create an environment of the same pH, but as the EtOH ratio increases, the replacement rate of H₂O increases and the time of the liquid phase step decreases. Although the yield came out slightly lower in the 10 mL/0.225 mL ratio condition than other conditions, the yield showed 80˜90% overall.

As the EtOH ratio increases, the maximum height at which the dried material is recovered increases gradually when recovering the dried material inside the supercritical dryer, and it can be seen that it almost rises to the top of the dryer under the 10 mL/0.475 mL ratio condition. If the EtOH ratio is higher than this, protein loss may occur. A continuous phase of CO₂ and EtOH flows over the surface of the culture solution. At this time, a concentration gradient of EtOH according to the height is formed due to the difference in density between CO₂ and EtOH. Since the attractive force of H₂O and EtOH is higher than that of CO₂ and EtOH, a concentration gradient is formed in the direction of decreasing EtOH concentration as the height increases near the culture solution. When EtOH decreases in the ternary system of CO₂+EtOH+H₂O, the amount of H₂O that can be included in a single phase decreases, so some mixed H₂O is separated into fine droplets. Since EtOH is lighter than CO₂, if it falls over a certain level from the surface of the culture solution, it is not affected by the attraction with H₂O. As the height increases, a concentration gradient is formed in the direction of increasing the concentration of EtOH. Therefore, to the point where the concentration gradient of EtOH starts to be affected by the density, fine H₂O droplets are formed and exist in a dispersed state. As the EtOH ratio increases, the corresponding point is formed at a higher position in the dryer. The H₂O droplets thus formed are eventually extracted by the CO₂+EtOH mixed fluid over time, and the protein particles dissolved in the H₂O droplets are precipitated and attached to the wall of the dryer. If the EtOH ratio is too high, a single phase of CO₂+EtOH+H₂O may be formed, which may damage yield. Therefore, 10 mL/0.475 mL is judged as the maximum CO₂/EtOH ratio value that can prevent the loss of protein due to the formation of a single phase before the complete substitution of H₂O. The total amount of VEGF shows the best results under the condition of a CO₂/EtOH ratio of 10 mL/0.475 mL, identical to the concentration of VEGF.

The structure retention rate of VEGF in aqueous phase of the dried sample is highest when the CO₂/EtOH ratio is 10/0.350 and decreases slightly again when decreasing, whereas one of the samples at 10/0.475 has a structure retention of 98.3%. One showed a large difference at 79.7%. Since the temperature, pressure, and total flow conditions of CO₂ are constant, the stress applied to the protein is similar, so it is not affected by the ratio of EtOH except for samples with low structural retention. The dried material of the freeze drying process has a significantly lower structural retention rate in the aqueous solution, so the total amount of VEGF decreases significantly after 14 days than the dried material of the supercritical drying process. The dried material through supercritical drying showed a higher VEGF structure retention rate than the sample (Control) without any drying process, which is thought to have been influenced by the presence or absence of an additive that has the potential to accelerate the hydrolysis rate of protein in the culture solution.

The results of a series of experiments show that the optimal CO₂/EtOH ratio in the supercritical drying process is 10 mL/min/0.475 mL/min, which can save process time.

In order to determine whether the residual solvent contained in the sample is at a level harmful to the human body under optimal conditions, the amount was measured through gas chromatography. According to the FDA, the intake limit of EtOH is 16,670 ppm, and the measurement result is 1.62 ppm, which is not harmful to the human body.

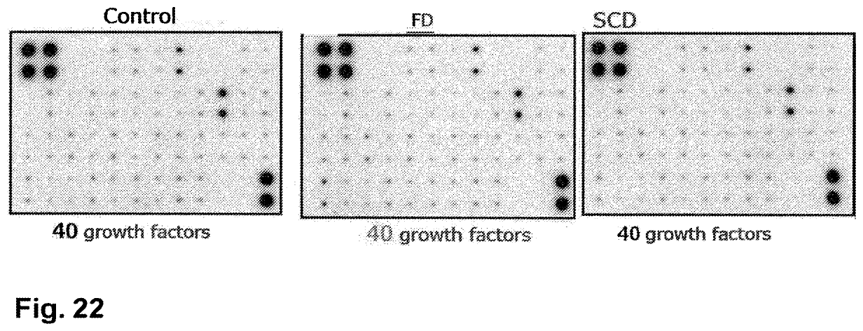

FIG. 22 shows the expression patterns of growth factors using a growth factor array of dried materials obtained under optimal conditions for each process.

Referring to FIG. 22, 40 growth factors were properly expressed for both supercritical drying (SCD) and freeze drying (FD).

The supercritical drying process performed for 160 minutes for the liquid phase step, 45 minutes for the supercritical phase step including temperature rise using a 0.02″ inner diameter nozzle at 25° C., 250 bar, a CO₂ flow rate of 10 mL/min, and an EtOH flow rate of 0.475 mL/min shows the best results in terms of the structure retention rate and total amount of VEGF in the initial stage, and the structure retention rate and total amount of VEGF when exposed to an aqueous solution environment for 14 days.

VEGF obtained through the supercritical drying process shows a relatively higher structural retention rate compared to VEGF obtained through the freeze drying process even when exposed to an aqueous environment for 14 days. Over time, supercritical drying is more advantageous than freeze drying in terms of the total amount of VEGF. Therefore, drying the protein through the supercritical drying process can inhibit protein damage than the freeze drying process.

Although the embodiments of the present invention have been disclosed for illustrative purposes, those skilled in the art will appreciate that the present invention may be embodied in other specific ways without changing the technical spirit or essential features thereof. Therefore, the embodiments disclosed in the present invention are not restrictive but are illustrative. The scope of the present invention is given by the claims, rather than the specification, and also contains all modifications within the meaning and range equivalent to the claims.

### INDUSTRIAL APPLICABILITY

Crystallization and supercritical drying of the culture solution according to embodiments of the present invention can dry the culture solution while preventing the structural stability and activity of the target material (dried material) dissolved in the culture solution from being denatured. The target material may be crystallized and dried. For example, the crystallization and supercritical drying may be used to crystallize and dry cells or proteins that are sensitive to temperature and need to maintain a microstructure, such as proteins. The crystallization and supercritical drying are superior to freeze drying in terms of process costs such as energy and process time. Mass production is possible by the crystallization and supercritical drying.

1. A method for crystallization and supercritical drying of culture solution to obtain target material by drying a culture solution containing a first solvent and the target material dissolved in the first solvent comprising:
crystallizing the target material by replacing the first solvent with a second solvent in the culture solution; and
changing a phase of the second solvent to a supercritical phase.

2. The method of claim 1, wherein the solvent replacing comprises replacing the first solvent with the second solvent and a third solvent, and
the phase changing comprises removing the third solvent.

3. The method of claim 2, wherein the second solvent has a critical temperature lower than the denaturation temperature of the target material.

4. The method of claim 2, wherein the third solvent is mixed with the first solvent and the second solvent.

5. The method of claim 2, wherein the first solvent comprises water, the second solvent comprises at least one of CO₂ and N₂O, and the third solvent comprises at least one of ethanol, acetone, N-methyl-2-pyrrolidone, and dimethyl sulfoxide.

6. The method of claim 1, wherein the target material maintains structural stability after the supercritical drying.

7. The method of claim 1, wherein the target material comprises cells.

8. The method of claim 1, wherein the target material comprises a protein.

9. The method of claim 1, further comprising vaporizing the second solvent in the supercritical phase.

10. The method of claim 1, wherein the method for crystallization and supercritical drying of culture solution is carried out at a temperature of 0˜40° C. and a pressure of 35˜500 bar.

11. An apparatus for crystallization and supercritical drying of culture solution to obtain target material by drying a culture solution containing a first solvent and the target material dissolved in the first solvent comprising:
a high pressure container to accommodate the culture solution;
a first supply unit connected to the high pressure container to supply a second solvent to the high pressure container;
a second supply unit connected to the high pressure container to supply a third solvent to the high pressure container;
a precooler disposed adjacent to the first supply unit to precool the second solvent discharged from the first supply unit; and
a preheater disposed adjacent to the high pressure container to preheat the second solvent and the third solvent supplied to the high pressure container.

12. The apparatus of claim 11, further comprising a capillary tube disposed in the high pressure container to transfer the second solvent and the third solvent into the high pressure container.

13. The apparatus of claim 11, further comprising a water tank accommodating the high pressure container and the preheater.

14. The apparatus of claim 11, further comprising a gas-liquid separator connected to the high pressure container to separate a fluid discharged from the high pressure container.

15. The apparatus of claim 11, further comprising a back pressure regulator disposed between the high pressure container and the gas-liquid separator to control the back pressure of the high pressure container.

16. The apparatus of claim 11, wherein the second solvent has a critical temperature lower than the denaturation temperature of the target material.

17. The apparatus of claim 11, wherein the third solvent is mixed with the first solvent and the second solvent.

18. The apparatus of claim 11, wherein the first solvent comprises water, the second solvent comprises at least one of CO₂ and N₂O, and the third solvent comprises at least one of ethanol, acetone, N-methyl-2-pyrrolidone, and dimethyl sulfoxide.
